SEQUENCE LISTING (4 sequences)

SEQ ID NO: 1 - protein, 574 aa, Homo sapiens
MASTEGANNMPKQVEVRMHDSHLGSEEPKHRHLGLRLCDKLGKNLLLTLTVFGVILGAVCGGLLRLASPIHPDVVMLIAFPGDILMRMLKMLILPLIISSLITGLSGLDAKASGRLGTRAMVYYMSTTIIAAVLGVILVLAIHPGNPKLKKQLGPGKKNDEVSSLDAFLDLIRNLFPENLVQACFQQIQTVTKKVLVAPPPDEEANATSAVVSLLNETVTEVPEETKMVIKKGLEFKDGMNVLGLIGFFIAFGIAMGKMGDQAKLMVDFFNILNEIVMKLVIMIMWYSPLGIACLICGKIIAIKDLEVVARQLGMYMVTVIIGLIIHGGIFLPLIYFVVTRKNPFSFFAGIFQAWITALGTASSAGTLPVTFRCLEENLGIDKRVTRFVLPVGATINMDGTALYEAVAAIFIAQMNGVVLDGGQIVTVSLTATLASVGAASIPSAGLVTMLLILTAVGLPTEDISLLVAVDWLLDRMRTSVNVVGDSFGAGIVYHLSKSELDTIDSQHRVHEDIEMTKTQSIYDDMKNHRESNSNQCVYAAHNSVIVDECKVTLAANGKSADCSVEEEPWKREK

SEQ ID NO: 2 - DNA, 1725 bp, Homo sapiens
atggcatctacggaaggtgccaacaatatgcccaagcaggtggaagtgcgaatgcacgacagtcatcttggctcagaggaacccaagcaccggcacctgggcctgcgcctgtgtgacaagctggggaagaatctgctgctcaccctgacggtgtttggtgtcatcctgggagcagtgtgtggagggcttcttcgcttggcatctcccatccaccctgatgtggttatgttaatagccttcccaggggatatactcatgaggatgctaaaaatgctcattctccctctaatcatctccagcttaatcacagggttgtcaggcctggatgctaaggctagtggccgcttgggcacgagagccatggtgtattacatgtccacgaccatcattgctgcagtactgggggtcattctggtcttggctatccatccaggcaatcccaagctcaagaagcagctggggcctgggaagaagaatgatgaagtgtccagcctggatgccttcctggaccttattcgaaatctcttccctgaaaaccttgtccaagcctgctttcaacagattcaaacagtgacgaagaaagtcctggttgcaccaccgccggacgaggaggccaacgcaaccagcgctgttgtctctctgttgaacgagactgtgactgaggtgccggaggagactaagatggttatcaagaagggcctggagttcaaggatgggatgaacgtcttaggtctgatagggtttttcattgcttttggcatcgctatggggaagatgggagatcaggccaagctgatggtggatttcttcaacattttgaatgagattgtaatgaagttagtgatcatgatcatgtggtactctcccctgggtatcgcctgcctgatctgtggaaagatcattgcaatcaaggacttagaagtggttgctaggcaactggggatgtacatggtaacagtgatcataggcctcatcatccacgggggcatctttctccccttgatttactttgtagtgaccaggaaaaaccccttctccttttttgctggcattttccaagcttggatcactgccctgggcaccgcttccagtgctggaactttgcctgtcacctttcgttgcctggaagaaaatctggggattgataagcgtgtgactagattcgtccttcctgttggagcaaccattaacatggatggtacagccctttatgaagcggtagccgccatctttatagcccaaatgaatggtgttgtcctggatggaggacagattgtgactgtaagcctcacagccaccctggcaagcgtcggcgcggccagtatccccagtgccgggctggtcaccatgctcctcattctgacagccgtgggcctgccaacagaggacatcagcctgctggtggctgtggactggctgctggacaggatgagaacttcagtcaatgttgtgggtgactcttttggggctgggatagtctatcacctctccaagtctgagctggataccattgactcccagcatcgagtgcatgaagatattgaaatgaccaagactcaatccatttatgatgacatgaagaaccacagggaaagcaactctaatcaatgtgtctatgctgcacacaactctgtcatagtagatgaatgcaaggtaactctggcagccaatggaaagtcagccgactgcagtgttgaggaagaaccttggaaacgtgagaaataa

SEQ ID NO: 3 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 4 - DNA, 3017 bp, Homo sapiens
ccgatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaagggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcactaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccacttcttcctgccacctgctgctgctgctgctgctaatcttcagggcactgctgctgcctttagtcgctgaggaaaaataaagacaaatgctgcgccctt

(19) United States
(12) Patent Application Publication
(10) Pub. No.: US 2012/0094295 A1
(43) Pub. Date: Apr. 19, 2012

(54) NEURODEGENERATIVE DISEASE DIAGNOSTIC COMPOSITIONS AND METHODS OF USE

(75) Inventors: Rita G. Sattler, Baltimore, MD (US); Jeffrey D. Rothstein, Baltimore, MD (US); Richard Chipkin, Baltimore, MD (US)

(73) Assignee: The Johns Hopkins University, Baltimore, MD (US)

(21) Appl. No.: 13/130,744

(22) PCT Filed: Nov. 20, 2009
(86) PCT No.: PCT/US09/06238

§ 371 Date: May 23, 2011

Related U.S. Application Data
(60) Provisional application No. 61/116,836, filed on Nov. 21, 2008.
(60) Provisional application No. 61/181,108, filed on May 26, 2009.

(51) Int. Cl. C12Q 1/68; G01N 33/53

(52) U.S. Cl. 435/6.12; 435/6.13; 435/7.1

(57) ABSTRACT

The invention generally provides diagnostics that employ biomarkers altered in neurodegenerative disease, as well as methods for the use of such markers in monitoring disease progression and identifying agents useful for the treatment of a neurodegenerative disease.

### CROSS-REFERENCE TO RELATED APPLICATION

This application claims the benefit of the following U.S. Provisional Application Nos. 61/116,836, filed Nov. 21, 2008, and 61/181,108, filed May 26, 2009, the entire contents of each of which are incorporated herein by reference.

### BACKGROUND OF THE INVENTION

Amyotrophic lateral sclerosis (ALS) is a fatal neurodegenerative disease. Clinical diagnosis of the disease is difficult, and typically takes many months to complete. Therefore, there is an unmet need for a means of rapidly diagnosing the disease and monitoring disease progression. Moreover, current methods of identifying agents likely to be useful for the treatment of ALS are inadequate and have failed to identify a single agent that is effective for the treatment of the disease. The identification of therapeutic agents useful for the treatment of neurodegeneration would be facilitated by the identification of biomarkers associated with neurodegenerative disease. The identification of such markers is made more difficult by a dearth of neural tissue samples. Therefore, the identification of peripheral markers associated with central nervous system (CNS) pathology is important to the development of assays for identification of therapeutic agents useful in treating diseases, particularly neurodegenerative diseases, affecting the CNS.

### SUMMARY OF THE INVENTION

As described below, the present invention provides compositions and methods for the diagnosis of amyotrophic lateral sclerosis (ALS) and other neurodegenerative diseases, as well as methods for monitoring disease progression and identifying agents useful for the treatment of neurodegenerative diseases.

In one aspect, the invention generally provides a method for identifying a subject (e.g., human) as having or having a propensity to develop a disease affecting the central nervous system, the method involving obtaining a nasal tissue sample from the subject; and detecting an alteration in an astrocytic polypeptide or polynucleotide in the tissue sample relative to a reference, thereby identifying the subject as having or having a propensity to develop a disease affecting the central nervous system.

In another aspect, the invention features a method for identifying a subject as having or having a propensity to develop a neurodegenerative disease associated with astrocyte dysfunction, the method involving obtaining a nasal tissue sample from the subject; and detecting an alteration (decrease or increase) in an EAAT2 or GFAP polypeptide or polynucleotide in the tissue sample relative to a reference, thereby identifying the subject as having or having a propensity to develop a neurodegenerative disease associated with astrocyte dysfunction.

In another aspect, the invention features a method for diagnosing a subject as having or having a propensity to develop amyotrophic lateral sclerosis, the method involving obtaining a nasal tissue sample from the subject; and detecting a reduction in an EAAT2 polypeptide or polynucleotide in the tissue sample relative to a reference, thereby identifying the subject as having or having a propensity to develop amyotrophic lateral sclerosis. In one embodiment, the method further involves identifying an increase in GFAP polypeptide or polynucleotide in the tissue sample relative to a reference.

In another aspect, the invention features a method for monitoring a disease affecting the central nervous system in a subject, the method involving obtaining a nasal tissue sample from the subject; and detecting an alteration in the level or function of an astrocytic polypeptide or polynucleotide in the tissue sample relative to a reference. In one embodiment, the reference is a subject sample obtained prior to treatment or at an earlier time point. In another embodiment, the astrocytic polypeptide or polynucleotide is an EAAT2 polypeptide or polynucleotide. In yet another embodiment, a reduction in EAAT2 expression relative to a reference indicates progression of the disease, and an increase in EAAT2 level indicates amelioration of the disease.

In yet another aspect, the invention features a method for detecting an agent's therapeutic efficacy in a subject having a disease affecting the central nervous system, the method involving obtaining a nasal tissue sample from the subject; and detecting an alteration in the level or function of an astrocytic polypeptide or polynucleotide in the tissue sample following treatment relative to a reference.

In another aspect, the invention features a method of identifying a candidate compound that ameliorates a neurodegenerative disorder, the method involving contacting an olfactory tissue cell that expresses an astrocytic polypeptide or polynucleotide with a candidate compound, and comparing the level of expression in the cell relative to the level present in a reference, where a compound that alters the level of the astrocytic polypeptide or polynucleotide is identified as ameliorating a neurodegenerative disorder. In one embodiment, the compound increases the expression of an astrocytic polypeptide or polynucleotide whose expression or activity is reduced in a neurodegenerative disease. In another embodiment, the compound increases the expression of a neuroprotective astrocytic protein. In yet another embodiment, the compound increases or decreases the level of astrocytic polypeptide or polynucleotide relative to an untreated control cell. In another embodiment, the cell is a mammalian (e.g., human, rodent) cell in vitro.

In another aspect, the invention features a method of assessing the efficacy of a treatment for a neurodegenerative disease in a subject, the method involving measuring an astrocytic marker in an olfactory tissue cell obtained from the subject; contacting the olfactory tissue cell with a candidate agent; and detecting an alteration in the marker level, where an alteration in the level of the marker is an indication that the treatment is efficacious for treating a neurodegenerative disease in the subject.

In another aspect, the invention features a method of assessing the efficacy of a treatment for a neurodegenerative disease in a subject, the method involving comparing: (i) the level of an astrocytic marker measured in a first olfactory tissue cell obtained from the subject before the treatment has been administered to the subject; and (ii) the level of the marker in a second olfactory epithelium biopsy sample obtained from the subject after the treatment has been administered to the subject, where an alteration in the level of the marker in the second sample relative to the first sample is an indication that the treatment is efficacious for treating a neurodegenerative disease in the subject. In one embodiment, the compound increases or decreases the level of astrocytic polypeptide or polynucleotide relative to an untreated control cell. In another embodiment, the compound increases the expression of an astrocytic polypeptide or polynucleotide whose expression or activity is reduced in a neurodegenerative disease. In yet another embodiment, the compound increases the expression of a neuroprotective astrocytic polypeptide.

In yet another aspect, the invention features a method of identifying a candidate compound that ameliorates a central nervous system disease or disorder, the method involving contacting olfactory tissue cell that expresses an astrocytic polypeptide or polynucleotide with a candidate compound, and comparing the level of expression of the polypeptide or polynucleotide in the cell contacted by the candidate compound with the level of expression in a corresponding control cell not contacted by the candidate compound, where an alteration in expression of the polypeptide or polynucleotide identifies the candidate compound as a candidate compound that ameliorates a central nervous system disease or disorder. In one embodiment, the compound increases the expression of an astrocytic polypeptide whose expression or activity is reduced in a central nervous system disease or disorder. In another embodiment, the compound reduces the expression of an astrocytic polypeptide whose expression or activity is increased in a central nervous system disease or disorder.

In another aspect, the invention features a method for increasing Glt-1 protein in a subject, the method involving orally administering to the subject an amount of thiamphenicol that is effective to specifically increase mRNA levels of astrocytic genes EAAT2 and GFAP in human astrocytes within the central nervous system. In one embodiment, the orally administered thiamphenicol is sufficient to generate cerebrospinal fluid levels between about 50 and 1000 ng/ml. In another embodiment, cerebrospinal fluid levels of thiamphenicol are at least about 75-125 ng/ml (e.g., 75, 80, 90, 95, 100, 105, 110, 115, 120, 125). In one embodiment, cerebrospinal fluid levels of thiamphenicol are at least about 100-350 ng/ml. In another embodiment, the reference was obtained from the subject prior to treatment or at an earlier time point during treatment.

In various embodiments of the above aspects or of any other aspect of the invention delineated herein, the polypeptide or polynucleotide is an astrocytic polypeptide or polynucleotide whose expression is altered in a CNS disease or disorder. In particular embodiments, the astrocytic polypeptide or polynucleotide is an EAAT2 or GFAP polypeptide or polynucleotide (e.g., human). In still other embodiments of the invention described herein, the tissue sample is obtained during an olfactory epithelial biopsy. In one embodiment, the tissue sample contains any one or more of olfactory receptor neurons, supporting cells, basal horizontal basal cells, global basal cells, sustentacular cells, olfactory ensheathing cells, fibroblasts, Bowman gland cells and blood vessel cells. In still other embodiments, the polypeptide or polynucleotide is any one or more of GFAP, S100β, MCT-4, EAAT2, EAAT1, Aquaporin-4; MCT-1; Connexin 30; Connexin 43 and OMP. In still other embodiments, the polypeptide or polynucleotide is an astrocytic polypeptide or polynucleotide (e.g., EAAT2) whose expression is altered in a CNS disease or disorder (e.g., amyotrophic lateral sclerosis, multiple sclerosis, epilepsy, stroke, Huntington's disease, noise-induced hearing loss, glioma, Parkinson's disease, obsessive-compulsive disorder, neuropathic pain, depression, and a mood disorder). In still other embodiments, the method detects the level of polypeptide or polynucleotide expression. For example, the alteration in polypeptide level is detected by fluorescence, protein activity, Western blot, enzyme-linked immunoassay, direct immunoassay, or radiometric assay. In another example, the alteration in polynucleotide level is detected by microarray analysis, Northern blot analysis, reverse transcriptase PCR, or quantitative real-time PCR. In still other embodiments of the above aspects, the reference is the level of polynucleotide or polypeptide present in a healthy control. In still other embodiments, the subject is a human subject (e.g., a subject identified as having or having a propensity to develop a CNS disease). In particular embodiments of the above aspects, the method detects a reduction in EAAT2 expression or function relative to a reference. In one embodiment, a compound that increases the level or function of EAAT2 is identified as having therapeutic efficacy in a subject.

The invention identifies biomarkers altered in neurodegenerative disease, and provides for the use of such biomarkers in diagnostic applications, drug screening, and patient monitoring. Other features and advantages of the invention will be apparent from the detailed description, and from the claims.

### DEFINITIONS

By “excitatory amino acid transporter 2 (EAAT2) polypeptide” is meant a protein substantially identical to the amino acid sequence of GenBank Accession No. NP_{—}004162, or a fragment thereof, and having at least one EAAT2 biological activity. EAAT2 biological activity includes glutamate transport activity, neuroprotective activity, or EAAT2 antibody binding activity. In one embodiment, an EAAT2 polypeptide has at least about 85% amino acid sequence identity to the following amino acid sequence:

| | |
|---|---|
| 1 | mastegannm pkqvevrmhd shlgseepkh rhlglrlcdk lgknllltlt vfgvilgavc |
| 61 | ggllrlaspi hpdvvmliaf pgdilmrmlk mlilpliiss litglsglda kasgrlgtra |
| 121 | mvyymsttii aavlgvilvl aihpgnpklk kqlgpgkknd evssldafld lirnlfpenl |
| 181 | vqacfqqiqt vtkkvlvapp pdeeanatsa vvsllnetvt evpeetkmvi kkglefkdgm |
| 241 | nvlgligffi afgiamgkmg dqaklmvdff nilneivmkl vimimwyspl giaclicgki |
| 301 | iaikdlevva rqlgmymvtv iigliihggi flpliyfvvt rknpfsffag ifqawitalg |
| 361 | tassagtlpv tfrcleenlg idkrvtrfvl pvgatinmdg talyeavaai fiaqmngvvl |
| 421 | dggqivtvsl tatlasvgaa sipsaglvtm lliltavglp tedisllvav dwlldrmrts |
| 481 | vnvvgdsfga givyhlskse ldtidsqhrv hediemtktq siyddmknhr esnsnqcvya |
| 541 | ahnsvivdec kvtlaangks adcsveeepw krek |

By “EAAT2 polynucleotide” is meant a nucleic acid molecule encoding an EAAT2 polypeptide or fragment thereof. An exemplary EAAT2 nucleic acid sequence (GenBank Accession No. NM_{—}004171) is provided below:

| | |
|---|---|
| 1 | atggcatcta cggaaggtgc caacaatatg cccaagcagg tggaagtgcg aatgcacgac |
| 61 | agtcatcttg gctcagagga acccaagcac cggcacctgg gcctgcgcct gtgtgacaag |
| 121 | ctggggaaga atctgctgct caccctgacg gtgtttggtg tcatcctggg agcagtgtgt |
| 181 | ggagggcttc ttcgcttggc atctcccatc caccctgatg tggttatgtt aatagccttc |
| 241 | ccaggggata tactcatgag gatgctaaaa atgctcattc tccctctaat catctccagc |
| 301 | ttaatcacag ggttgtcagg cctggatgct aaggctagtg gccgcttggg cacgagagcc |
| 361 | atggtgtatt acatgtccac gaccatcatt gctgcagtac tgggggtcat tctggtcttg |
| 421 | gctatccatc caggcaatcc caagctcaag aagcagctgg ggcctgggaa gaagaatgat |
| 481 | gaagtgtcca gcctggatgc cttcctggac cttattcgaa atctcttccc tgaaaacctt |
| 541 | gtccaagcct gctttcaaca gattcaaaca gtgacgaaga aagtcctggt tgcaccaccg |
| 601 | ccggacgagg aggccaacgc aaccagcgct gttgtctctc tgttgaacga gactgtgact |
| 661 | gaggtgccgg aggagactaa gatggttatc aagaagggcc tggagttcaa ggatgggatg |
| 721 | aacgtcttag gtctgatagg gtttttcatt gcttttggca tcgctatggg gaagatggga |
| 781 | gatcaggcca agctgatggt ggatttcttc aacattttga atgagattgt aatgaagtta |
| 841 | gtgatcatga tcatgtggta ctctcccctg ggtatcgcct gcctgatctg tggaaagatc |
| 901 | attgcaatca aggacttaga agtggttgct aggcaactgg ggatgtacat ggtaacagtg |
| 961 | atcataggcc tcatcatcca cgggggcatc tttctcccct tgatttactt tgtagtgacc |
| 1021 | aggaaaaacc ccttctcctt ttttgctggc attttccaag cttggatcac tgccctgggc |
| 1081 | accgcttcca gtgctggaac tttgcctgtc acctttcgtt gcctggaaga aaatctgggg |
| 1141 | attgataagc gtgtgactag attcgtcctt cctgttggag caaccattaa catggatggt |
| 1201 | acagcccttt atgaagcggt agccgccatc tttatagccc aaatgaatgg tgttgtcctg |
| 1261 | gatggaggac agattgtgac tgtaagcctc acagccaccc tggcaagcgt cggcgcggcc |
| 1321 | agtatcccca gtgccgggct ggtcaccatg ctcctcattc tgacagccgt gggcctgcca |
| 1381 | acagaggaca tcagcctgct ggtggctgtg gactggctgc tggacaggat gagaacttca |
| 1441 | gtcaatgttg tgggtgactc ttttggggct gggatagtct atcacctctc caagtctgag |
| 1501 | ctggatacca ttgactccca gcatcgagtg catgaagata ttgaaatgac caagactcaa |
| 1561 | tccatttatg atgacatgaa gaaccacagg gaaagcaact ctaatcaatg tgtctatgct |
| 1621 | gcacacaact ctgtcatagt agatgaatgc aaggtaactc tggcagccaa tggaaagtca |
| 1681 | gccgactgca gtgttgagga agaaccttgg aaacgtgaga aataa |

By “glial fibrillary acidic protein (GFAP) polypeptide” is meant a protein substantially identical to the amino acid sequence of GenBank Accession No. AAB22581, or a fragment thereof, and having at least one GFAP biological activity. An exemplary GFAP biological activity is supporting astrocyte cell structure, movement, or GFAP specific antibody binding. In one embodiment, a GFAP polypeptide has at least about 85% amino acid sequence identity to the following amino acid sequence:

| | |
|---|---|
| 1 | merrritsaa rrsyvssgem mvgglapgrr lgpgtrlsla rmppplptrv dfslagalna |
| 61 | gfketraser aemmelndrf asyiekvrfl eqqnkalaae lnqlrakept kladvyqael |
| 121 | relrlrldql tansarleve rdnlaqdlat vrqklqdetn lrleaennla ayrqeadeat |
| 181 | larldlerki esleeeirfl rkiheeevre lqeqlarqqv hveldvakpd ltaalkeirt |
| 241 | qyeamassnm heaeewyrsk fadltdaaar naellrqakh eandyrrqlq sltcdleslr |
| 301 | gtneslerqm reqeerhvre aasyqealar leeegqslkd emarhlqeyq dllnvklald |
| 361 | ieiatyrkll egeenritip vqtfsnlqir etsldtksys eghlkrnivv ktvemrdgev |
| 421 | ikeskqehkd vm |

By “GFAP polynucleotide” is meant a nucleic acid molecule encoding a GFAP polypeptide or fragment thereof. An exemplary GFAP polynucleotide sequence is provided below (Genbank Accession No. J04569).

| | |
|---|---|
| 1 | ccgatggaga ggagacgcat cacctccgct gctcgccgct cctacgtctc ctcaggggag |
| 61 | atgatggtgg ggggcctggc tcctggccgc cgtctgggtc ctggcacccg cctctccctg |
| 121 | gctcgaatgc cccctccact cccgacccgg gtggatttct ccctggctgg ggcactcaat |
| 181 | gctggcttca aggagacccg ggccagtgag cgggcagaga tgatggagct caatgaccgc |
| 241 | tttgccagct acatcgagaa ggttcgcttc ctggaacagc aaaacaaggc gctggctgct |
| 301 | gagctgaacc agctgcgggc caaggagccc accaagctgg cagacgtcta ccaggctgag |
| 361 | ctgcgagagc tgcggctgcg gctcgatcaa ctcaccgcca acagcgcccg gctggaggtt |
| 421 | gagagggaca atctggcaca ggacctggcc actgtgaggc agaagctcca ggatgaaacc |
| 481 | aacctgaggc tggaagccga gaacaacctg gctgcctata gacaggaagc agatgaagcc |
| 541 | accctggccc gtctggatct ggagaggaag attgagtcgc tggaggagga gatccggttc |
| 601 | ttgaggaaga tccacgagga ggaggttcgg gaactccagg agcagctggc ccgacagcag |
| 661 | gtccatgtgg agcttgacgt ggccaagcca gacctcaccg cagccctgaa agagatccgc |
| 721 | acgcagtatg aggcaatggc gtccagcaac atgcatgaag ccgaagagtg gtaccgctcc |
| 781 | aagtttgcag acctgacaga cgctgctgcc cgcaacgcgg agctgctccg ccaggccaag |
| 841 | cacgaagcca acgactaccg gcgccagttg cagtccttga cctgcgacct ggagtctctg |
| 901 | cgcggcacga acgagtccct ggagaggcag atgcgcgagc aggaggagcg gcacgtgcgg |
| 961 | gaggcggcca gttatcagga ggcgctggcg cggctggagg aagaggggca gagcctcaag |
| 1021 | gacgagatgg cccgccactt gcaggagtac caggacctgc tcaatgtcaa gctggccctg |
| 1081 | gacatcgaga tcgccaccta caggaagctg ctagagggcg aggagaaccg gatcaccatt |
| 1141 | cccgtgcaga ccttctccaa cctgcagatt cgagaaacca gcctggacac caagtctgtg |
| 1201 | tcagaaggcc acctcaagag gaacatcgtg gtgaagaccg tggagatgcg ggatggagag |
| 1261 | gtcattaagg agtccaagca ggagcacaag gatgtgatgt gaggcaggac ccacctggtg |
| 1321 | gcctctgccc cgtctcatga ggggcccgag cagaagcagg atagttgctc cgcctctgct |
| 1381 | ggcacatttc cccagacctg agctccccac caccccagct gctcccctcc ctcctctgtc |
| 1441 | cctaggtcag cttgctgccc taggctccgt cagtatcagg cctgccagac ggcacccacc |
| 1501 | cagcacccag caactccaac taacaagaaa ctcaccccca agggcagtct ggaggggcat |
| 1561 | ggccagcagc ttgcgttaga atgaggagga aggagagaag gggaggaggg cggggggcac |
| 1621 | ctactacatc gccctccaca tccctgattc ctgttgttat ggaaactgtt gccagagatg |
| 1681 | gaggttctct cggagtatct gggaactgtg cctttgagtt tcctcaggct gctggaggaa |
| 1741 | aactgagact cagacaggaa agggaaggcc ccacagacaa ggtagccctg gccagaggct |
| 1801 | tgttttgtct tttggttttt atgaggtggg atatccctat gctgcctagg ctgaccttga |
| 1861 | actcctgggc tcaagcagtc tacccacctc agcctcctgt gtagctggga ttatagattg |
| 1921 | gagccaccat gcccagctca gagggttgtt ctcctagact gaccctgatc agtctaagat |
| 1981 | gggtggggac gtcctgccac ctggggcagt cacctgccca gatcccagaa ggacctcctg |
| 2041 | agcgatgact caagtgtctc agtccacctg agctgccatc cagggatgcc atctgtgggc |
| 2101 | acgctgtggg caggtgggag cttgattctc agcacttggg ggatctgttg tgtacgtgga |
| 2161 | gagggatgag gtgctgggag ggatagaggg gggctgcctg gcccccagct gtgggtacag |
| 2221 | agaggtcaag cccaggagga ctgccccgtg cagactggag gggacgctgg tagagatgga |
| 2281 | ggaggaggca attgggatgg cactaggcat acaagtaggg gttgtgggtg accagttgca |
| 2341 | cttggcctct ggattgtggg aattaaggaa gtgactcatc ctcttgaaga tgctgaaaca |
| 2401 | ggagagaaag gggatgtatc catgggggca gggcatgact ttgtcccatt tctaaaggcc |
| 2461 | tcttccttgc tgtgtcatac caggccgccc cagcctctga gcccctggga ctgctgcttc |
| 2521 | ttaaccccag taagccactg ccacacgtct gaccctctcc accccatagt gaccggctgc |
| 2581 | ttttccctaa gccaagggcc tcttgcggtc ccttcttact cacacacaaa atgtacccag |
| 2641 | tattctaggt agtgccctat tttacaattg taaaactgag gcacgagcaa agtgaagaca |
| 2701 | ctggctcata ttcctgcagc ctggaggccg ggtgctcagg gctgacacgt ccaccccagt |
| 2761 | gcacccactc tgctttgact gagcagactg gtgagcagac tggtgggatc tgtgcccaga |
| 2821 | gatgggactg ggagggccca cttcagggtt ctcctctccc ctctaaggcc gaagaagggt |
| 2881 | ccttccctct ccccaagact tggtgtcctt tccctccact tcttcctgcc acctgctgct |
| 2941 | gctgctgctg ctaatcttca gggcactgct gctgccttta gtcgctgagg aaaaataaag |
| 3001 | acaaatgctg cgccctt |

By “astrocyte dysfunction” is meant any undesirable alteration in astrocyte cellular function, gene expression or astrocytic protein function. An undesirable alteration in EAAT2 or GFAP protein or gene expression or biological activity is indicative of astrocyte dysfunction. Other indicators of astrocyte dysfunction include a loss or reduction in the maintenance of neuronal metabolism, neuronal health, neurotransmitter synthesis, an increase in neuronal cell death, or any other alteration indicative of neurodegeneration.

By “disease affecting the central nervous system” is meant any pathology that reduces the normal function or integrity of a CNS cell or tissue. Such cells and tissues include but are not limited to astrocytes, neurons, blood vessels, or any other cell contained within the CNS.

By “neurodegenerative disease” is meant a reduction in neuronal cell structure or function or a reduction in the structure or function of a myelin sheath. An increase in cell death is indicative of neurodegenerative disease.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10%-100% change in expression levels (e.g., 10, 20, 30, 40, 50, 60, 75, 80, 85, 90, 95, 100%) change in expression levels.”

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface, and therefore, function as probes.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte to be detected.

“Diagnostic” means identifying the presence or nature of a pathologic condition, i.e., a neurodegenerative disease. While a particular diagnostic method may not provide a definitive diagnosis of a condition, it suffices if the method provides a positive indication that aids in diagnosis.

The phrase “differentially present” refers to differences in the quantity and/or the frequency of a marker present in a sample taken from subjects having a disease as compared to a control subject. A marker can be differentially present in terms of quantity, frequency or both. A polypeptide is differentially present between two samples if the amount of the polypeptide in one sample is statistically significantly different from the amount of the polypeptide in the other sample. Alternatively or additionally, a polypeptide is differentially present between two sets of samples if the frequency of detecting the polypeptide in a diseased subjects' samples is statistically significantly higher or lower than in the control samples.

By “disease” is meant any condition or disorder that damages or interferes with the normal function of a cell, tissue, or organ.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a neurodegenerative disorder characterized by the methods delineated herein. In addition, the methods of the invention provide a facile means to identify therapies that are safe for use in eukaryotic host organisms. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “function” is meant any biological activity of a polypeptide or polynucleotide.

As used herein, the terms “gene” and “recombinant gene” refer to nucleic acid molecules comprising an open reading frame encoding a marker protein.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “microarray” is meant an organized collection of at least two nucleic acid molecules or polypeptides affixed to a solid support. In some embodiments, a nucleic acid microarray is composed of oligonucleotides having at least a portion (e.g., 10, 15, 18, 20, 21, 22, 23, 24, 25, 26, 27, 28, 29, 30, 40, 50, 60, 70, 80, 90, or 100 nucleotides) of two or more nucleic acid sequences delineated herein (e.g., GFAP, S100β, MCT-4, EAAT2, EAAT1). A polypeptide microarray contains at least a polypeptide (e.g., 10, 20, 30, 40, 50, 75, or 100 amino acids) delineated herein. A microarray contains at least 1, 2, 3, 4, 5, 6 polypeptide or nucleic acid molecules delineated herein.

“Monitoring” refers to recording changes in a continuously varying parameter (e.g. monitoring progression of a disease).

As used herein, “obtaining” as in “obtaining an agent” includes synthesizing, purchasing, or otherwise acquiring the agent.

“Protein biochip” refers to a biochip adapted for the capture of polypeptides.

By “reference” is meant a standard or control condition.

As used herein, “sample” or “biological sample” refers to anything, which may contain an analyte (e.g., polypeptide, polynucleotide, or fragment thereof) for which an analyte assay is desired. The sample may be a biological sample, such as a biological fluid or a biological tissue. In one embodiment, a biological sample is obtained from olfactory epithelium. Such a sample may include diverse cell types, including connective, epithelium, muscle and nerve tissues. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of marker-detected subjects with a particular disease.

As used herein, the term “specificity” is the percentage of subjects correctly identified as having a particular disease i.e., normal or healthy subjects. For example, the specificity is calculated as the number of subjects with a particular disease as compared to non-cancer subjects (e.g., normal healthy subjects).

By “subject” is meant a mammal, including, but not limited to, a human or non-human mammal, such as a bovine, equine, canine, ovine, rodent, or feline.

“Solid support” refers to a solid material, which can be derivatized with, or otherwise attached to, a capture reagent. Exemplary solid supports include probes, microtiter plates and chromatographic resins.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A and 1B are Western blots and a graph, respectively, showing that EAAT2/GLT-1 protein and mRNA is expressed in nasal tissue samples from rodents and humans. FIG. 1A provides a Western blot analysis of human and rodent nasal olfactory mucosa tissue showed expression of EAAT2/GLT-1 protein, both multimer and monomer complexes. The protein expression level in the nasal tissue is lower than it is in spinal cord tissue. FIG. 1B provides a quantitative mRNA analysis, which confirms that expression of GLT-1 in the olfactory epithelium of mice (OE) is lower than GLT-1 expression in cortex or spinal cord.

FIGS. 2A-2D are micrographs showing that EAAT2/GLT-1 is expressed in the basal cell layer of the human olfactory epithelium. FIG. 2A shows hematoxylin and eosin (H&E) staining of human nasal biopsy samples. This analysis shows the anatomy of the olfactory mucosa. The following abbreviations are used throughout the Figures: olfactory epithelium (OE), lamina propria (LP), Bowman's gland (BG), blood vessels (BV) in the LP, and axon bundles (AB) in the LP. FIG. 2B shows immunostaining results for the basal cell marker p75NGFR in nasal biopsy samples. FIG. 2C shows immunostaining results for the glutamate transporter EAAT2/GLT-1. FIG. 2D shows immunostaining results for astrocyte marker GFAP. EAAT2/GLT-1 immunostaining co-localizes with basal cell marker p75NGFR. GFAP is highly expressed in sustentacular cells of the OE (FIG. 2D, arrowhead), as well as in ensheathing cells surrounding axon bundles in the LP (FIG. 2D, Arrow).

FIGS. 3A-3D are graphs showing that a thiamphenicol (TAP)-induced GLT-1 protein increase results in enhanced glutamate uptake and neuroprotection. FIG. 3A is a graph showing EAAT2 promoter activation. EAAT2 promoter activation was examined by measuring luciferase activity using a reporter gene construct driven by a 2.5 kB promoter fragment of EAAT2 (Rothstein et al., Nature. 2005 Jan. 6; 433(7021):73-7). Human immortalized astrocytes were transiently transfected with this reporter gene construct and treated for 72 hours with a concentration range of Thiamphenicol. FIG. 3B is a graph showing GLT-1 protein levels in primary mouse cultures of neurons and astrocytes that were treated with Thiamphenicol for 72 hours. GLT-1 protein was measured using standard western blot analysis and revealed a dose-dependent increase of GLT-1 with TAP treatment. FIG. 3C shows glutamate-uptake results in primary mouse cultures of neurons and astrocytes that were treated with Thiamphenicol for 72 hours. Glutamate-uptake was measured using standard techniques. TAP dose-dependently increased glutamate uptake in these cultures. FIG. 3D is a graph showing cell death results in primary mouse cultures of neurons and astrocytes that were treated with Thiamphenicol for 72 hours. Cells were then exposed to an oxygen-glucose-free environment for 1.5 hours (Goldberg et al). After the oxygen-glucose deprivation, cells were washed back into normal artificial cerebral spinal fluid (ACSF) solution and maintained for another 23 hours at 37 C, 5% CO₂. Neuronal cell death was determined by measuring lactose dehydrogenase (LDH) release. Cells treated with MK-801, 6-cyano-7-nitroquinoxaline-2,3-dione), (CNQX) and nimodipine (MCN) were used as a positive control for neuroprotective activity. TAP showed dose-dependent neuroprotection in the oxygen-glucose deprivation (OGD) assay using the present in vitro culture model.

FIGS. 4A-4C are graphs showing that thiamphenicol increased GLT-1 mRNA, protein and glutamate uptake in vivo. Wild-type (WT) mice were treated for 10 days with 12.5 or 25 mg/kg/day thiamphenicol (TAP) via oral delivery. TAP dose-dependently increased GLT-1 protein (as measured in brain cortex) (FIG. 4A) and mRNA (FIG. 4B). Synaptosomal preparations obtained from brain cortex showed a TAP-dependent increase in functional glutamate transport (FIG. 4C).

FIG. 5 is a Western blot showing that the GLT-1 upregulating agent Thiamphenicol increased GLT-1 protein levels in mouse olfactory epithelium. Wild-type (WT) mice were treated with Thiamphenicol for 10 days (25 mg/kg/day, intraperitoneal administration). Olfactory epithelial tissue was isolated and probed for GLT-1 protein expression using standard Western Blot analysis. TAP increased GLT-1 protein levels in comparison to saline treated animals.

FIG. 6 is a graph showing the linear correlation between plasma concentrations of TAP and CSF levels. Thiamphenicol plasma levels were correlated with the levels of Thiamphenicol in CSF taken from the volunteers on the second last day of drug treatment. The data show a nice correlation between the two readouts.

FIGS. 7A-7E are graphs showing that the GLT-1 upregulating agent thiamphenicol increased EAAT2/GLT-1 mRNA levels in human olfactory epithelium. Healthy volunteers were treated with Thiamphenicol for 14 days (0; 750 mg; 1500 mg once a day, oral). Nasal biopsies were performed the day before the beginning of drug treatment (day −1) and on day 14. mRNA levels of several genes were quantified by qPCR analysis. Data are presented as fold increases in mRNA levels after drug treatment for each individual patient. FIGS. 7A and 7B show a positive change in astrocytic genes EAAT2/GLT-1 and GFAP. This confirmed a positive trend towards increased gene expression levels while non-astrocytic genes EAAT2/EAAT1 (FIG. 7C), OMP (FIG. 7D) and MCT-4 (FIG. 7E) showed no change in gene expression levels after drug treatment.

### DETAILED DESCRIPTION OF THE INVENTION

The invention features compositions and methods useful for identifying patients as having a central nervous system (CNS) disorder or disease, for monitoring the progression of the disease in such patients, and for identifying therapeutic agents useful for the treatment of a central nervous system disorder or disease.

The invention is based, at least in part, on the discovery that astrocytic proteins are expressed in olfactory epithelium, and that agents that alter the expression of astrocytic proteins in the CNS are also effective at altering levels of such proteins in olfactory epithelium. Thus, the olfactory epithelium, which is readily accessible for biopsy, can be used to monitor astrocytic protein expression for the diagnosis of a neurodegenerative disease, or to monitor disease progression. This provides an important advance given that astrocytes found in the central nervous system are comparatively inaccessible for the diagnosis and monitoring of disease. Astrocytic proteins present in nasal epithelium include but are not limited to GFAP, EAAT2/GLT-1, S100β, Aquaporin-4; MCT-1; Connexin 30; and Connexin 43. EEAT2 is a glutamate transporter protein that is downregulated and/or dysfunctional in many chronic and acute central nervous system disorders or diseases. For example, alterations in EAAT2 levels are associated with amyotrophic lateral sclerosis, muscular sclerosis, epilepsy, stroke, Huntington's disease, and mood disorders, such as depression. EAAT2 has been implicated in excitotoxic neuronal cell death during disease. To test whether this biomarker responds to drug therapy that increases levels of the brain glutamate transporter protein GLT-1, GLT-1 levels were compared in mice that were untreated relative to mice treated with thiamphenicol. RNA and protein analysis confirmed an effect of drug treatment on the expression of astroglial protein GLT-1 in olfactory tissue. Thus, the invention provides methods for detecting alterations in EAAT2/GLT-1 and other astrocytic proteins in nasal biopsy samples. Such methods are useful for the diagnosis and monitoring of ALS. Cells obtained during a nasal biopsy are also useful for the evaluation of drugs that maybe useful for the treatment or prevention of ALS and other neurodegenerative disease.

### Markers

The present invention provides markers expressed in olfactory epithelium, whose expression or function is altered in a subject suffering from a disease that affects the central nervous system. Such markers, which include astrocytic polypeptides and nucleotides, are identified from comparisons of protein and gene expression profiles from human subjects. Astrocytic polypeptides and polynucleotides expressed in olfactory epithelium include, but are not limited to, GFAP, S100β, GLT-1/EAAT2, Aquaporin-4; MCT-1; Connexin 30; Connexin 43. In other embodiments, the invention further provides for the use of one or more of the following proteins: GFAP, S100β, MCT-4, EAAT2, EAAT1, OMP, glutamate receptors subtypes, nerve growth factor receptor, neurotransmitters GABA and/or glutamate in compositions and methods of the invention. In particular, the invention provides that such markers used individually or in combination with other markers, provide a method of diagnosing and/or monitoring a neurodegenerative disease or evaluating the efficacy of a therapeutic agent in a subject having a neurodegenerative disease. Neurodegenerative diseases include, but are not limited to amyotrophic lateral sclerosis, muscular sclerosis, epilepsy, stroke, Huntington's disease, and mood disorders, such as depression. In particular embodiments, the invention provides for the use of EAAT2.

Markers that are differentially present in samples of subjects having a neurodegenerative disease and control subjects find application in methods and kits for determining neurodegenerative disease status. Accordingly, methods are provided for identifying a neurodegenerative disease (e.g., amyotrophic lateral sclerosis, muscular sclerosis, epilepsy, stroke, Huntington's disease, mood disorders, depression) in a subject involve detecting a differential presence of a biomarker in subjects with a neurodegenerative disease in a biological sample obtained from a sample (e.g., an olfactory epithelium nasal biopsy) of the subject. The amount of one or more biomarkers found in a test sample compared to a control, or the presence or absence of one or more markers in the test sample provides useful information regarding the neurodegenerative disease status of the patient.

The markers can be measured in different types of biological samples. Preferably, the sample is obtained during a biopsy of olfactory epithelium. In one embodiment, preparation of the tissue sample for analysis involves homogenization and/or lysis of the tissue sample. In another embodiment, preparation of the tissue sample may involve fractionation of the sample and collection of fractions determined to contain one or more biomarkers. Methods of pre-fractionation include, for example, size exclusion chromatography, ion exchange chromatography, heparin chromatography, affinity chromatography, sequential extraction, gel electrophoresis and liquid chromatography. The biomarkers also may be modified prior to detection.

Proteins may be present in a sample in one or more modified forms. Modified forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, and acetylation. Modified forms of any marker of this invention also may be used, themselves, as biomarkers. In certain cases the modified forms may exhibit better discriminatory power in diagnosis than an unmodified form.

In certain embodiments, it may be helpful to purify a marker detected by the methods disclosed herein prior to subsequent analysis. Nearly any means known to the art for the purification and separation of small molecular weight substances, e.g., anion or cation exchange chromatography, gas chromatography, liquid chromatography or high pressure liquid chromatography may be used. Methods of selecting suitable separation and purification techniques and means of carrying them out are known in the art (see, e.g., Labadarious et. al., J. Chromatography (1984) 310:223-231, and references cited therein; and Shahrokhin and Gehrke, J. Chromatography (1968) 36:31-41, and Niessen J. Chromatography (1998) 794:407-435).

### Detection Methods

The invention provides methods of detecting one or more markers associated with a neurodegenerative disease or control markers in a tissue sample obtained from a subject. Although the invention specifically describes the use of immunoassays and PCR-based assays to identify the expression of certain polypeptides (e.g., GFAP, S100β, GLT-1/EAAT2), the invention is not so limited. One of skill in the art will recognize that any suitable method can be used to detect the markers described herein. Successful practice of the invention can be achieved with one or a combination of methods that can detect and/or quantify the markers. Such methods include, without limitation, hybridization-based methods including those employed in biochip arrays, mass spectrometry (e.g., laser desorption/ionization mass spectrometry), fluorescence (e.g. sandwich immunoassay), surface plasmon resonance, ellipsometry and atomic force microscopy. Methods may further include, by one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), atmospheric pressure photoionization mass spectrometry (APPI-MS), quadrupole mass spectrometry, fourier transform mass spectrometry (FTMS), and ion trap mass spectrometry. Detection methods may include use of a biochip array.

### Microarrays

As described herein, collections of polypeptides or polynucleotides may be used to identify expression profiles that are associated with neurodegenerative disease. These collections preferably include astrocytic polypeptides or polynucleotides (e.g., GFAP, S100β, GLT-1/EAAT2). Optionally, collections of the invention also include non-astrocytic proteins (e.g., EAAT1, OMP, MCT-4). Such nucleic acid molecules or polypeptides of the invention are useful as hybridizable array elements in a microarray. Polypeptides and polynucleotides useful in arrays of the invention include but are not limited to one, two, three, four, five, six, seven, or eight, of the following: GFAP, S100β, MCT-4, EAAT2, EAAT1, OMP, glutamate receptors subtypes, nerve growth factor receptors, Aquaporin-4, MCT-1, Connexin 30, and Connexin 43. The array elements are organized in an ordered fashion such that each element is present at a specified location (i.e., an addressable location) on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

Nucleic Acid Microarrays

To produce a nucleic acid microarray, oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a tissue sample (e.g. a tissue sample obtained by biopsy) or a bodily fluid sample (such as blood, cerebrospinal fluid, phlegm, saliva, or urine). For some applications, cultured cells (e.g., cells cultured from olfactory epithelium) or other tissue preparations may be used. The mRNA is isolated according to standard methods, and cDNA is produced and used as a template to make complementary RNA suitable for hybridization. In one embodiment, the RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, salt concentration, organic solvent percentage, and temperature conditions are varied to achieve the desired stringency. Varying additional parameters, such as hybridization time, the concentration of detergent, e.g., sodium dodecyl sulfate (SDS), and the inclusion or exclusion of carrier DNA, are well known to those skilled in the art. Useful variations will be readily apparent to those skilled in the art. A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

Marker proteins associated with a neurodegenerative disease, such as those described herein, may be analyzed using protein microarrays. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins are spotted on a substrate using any convenient method known to the skilled artisan (e.g., by hand or by inkjet printer). Preferably, such methods retain the biological activity or function of the protein bound to the substrate (Ge et al., supra; Zhu et al., supra).

The protein microarray is hybridized with a detectable probe. Such probes can be polypeptide, nucleic acid, or small molecules. For some applications, polypeptide and nucleic acid probes are derived from a biological sample taken from a patient, such as a homogenized tissue sample (e.g. a tissue sample obtained by biopsy). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

The biochip surfaces may, for example, be ionic, anionic, hydrophobic; comprised of immobilized nickel or copper ions, comprised of a mixture of positive and negative ions; and/or comprised of one or more antibodies, single or double stranded nucleic acids, proteins, peptides or fragments thereof, amino acid probes, or phage display libraries. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Markers may be captured with capture reagents immobilized to a solid support, such as a biochip, a multiwell microtiter plate, a resin, or nitrocellulose membranes that are subsequently probed for the presence of proteins. Capture can be on a chromatographic surface or a biospecific surface. For example, a sample containing the markers, such as a lysate obtained from a tissue sample, may be contacted with the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry, and in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure an astrocytic marker present in olfactory epithelium. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapour phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Frazingham, Mass., USA) and are described in the literature, e.g. M. Kussmann and P. Roepstorff, cited above.

### Diagnostics

Expression levels of particular nucleic acids or polypeptides may be correlated with a particular disease state, and thus are useful in diagnosis. In one embodiment, a patient having a neurodegenerative disease will show an alteration in the expression of an astrocytic polypeptide or polynucleotide (e.g., an EAAT2 polypeptide or polynucleotide) delineated herein. In another embodiment, a patient having a neurodegenerative disease will have a particular expression profile that includes significantly altered expression of two or more astrocytic polypeptides and/or polynucleotides (e.g., GFAP, S100β, GLT-1/EAAT2). Alterations in gene expression are detected using methods known to the skilled artisan and described herein.

In one embodiment, oligonucleotides or longer fragments derived from any of the nucleic acid sequences described herein may be used as targets in a microarray. The microarray is used to assay the expression level of large numbers of genes simultaneously and to identify alterations in the level of expression. If desired, the methods of the invention are used to characterize genetic variants, mutations, and polymorphisms. Such information can be used to diagnose a neurodegenerative disease or a subject having a propensity to develop such a condition.

In one embodiment, an alteration in the expression of a nucleic acid sequence described herein is detected using quantitative real-time reverse transcription PCR (qRT-PCR) to detect changes in gene expression. qRT-PCR methods are known in the art and are described herein. In another embodiment, an antibody that specifically binds a polypeptide described herein may be used for the diagnosis of a neurodegenerative disease. A variety of protocols for measuring an alteration in the expression of such polypeptides are known, including immunological methods (such as ELISAs and RIAs), and provide a basis for diagnosing a neurodegenerative disease. Again, an alteration in the level or function of an astrocytic polypeptide is diagnostic of a patient having a neurodegenerative disease (e.g., amyotrophic lateral sclerosis, muscular sclerosis, epilepsy, stroke, Huntington's disease, and mood disorders, such as depression).

In yet another embodiment, hybridization with PCR probes that are capable of detecting at least one of the polynucleotide sequences delineated herein, including genomic sequences, or closely related molecules, may be used to hybridize to a nucleic acid sequence derived from a patient having a neurodegenerative disease. In yet another approach, humans may be diagnosed for a propensity to develop a neurodegenerative disease by direct analysis of polypeptide or polynucleotide expression, or analysis of the sequence of a polynucleotide delineated herein.

In additional embodiment of the methods of the present invention, multiple markers are measured. The use of multiple markers increases the predictive value of the test and provides greater utility in diagnosis, toxicology, patient stratification and patient monitoring. The process detects expression profiles formed by the analysis of multiple markers. Such analysis may improve the sensitivity and specificity of clinical proteomics for predictive medicine. Subtle variations in data from clinical samples indicate that certain patterns of protein expression can predict phenotypes such as the presence or absence of a certain disease, a particular stage of disease progression, or a positive or adverse response to drug treatments.

Data generated by detection of markers can be analyzed using any suitable means. In one embodiment, data is analyzed with the use of a programmable digital computer. The computer program generally contains a readable medium that stores data. This data can indicate the number of markers detected, including the strength of the signal generated by each marker. Data analysis can include the steps of determining signal strength of a marker detected. When the sample is measured and data is generated, the data is then analyzed by a computer software program

As indicated above, the invention provides methods for aiding a human neurodegenerative disease diagnosis using one or more markers, as specified herein. These markers can be used alone, in combination with other markers in any set, or with entirely different markers in aiding human neurodegenerative disease diagnosis. The markers are differentially present in samples of a subject having or having a propensity to develop a neurodegenerative disease and a normal subject in whom neurodegenerative disease is undetectable. For example, some of the markers are expressed at an elevated level and/or are present at a higher frequency in human neurodegenerative disease subjects than in normal subjects, while some of the markers are expressed at a decreased level and/or are present at a lower frequency in human neurodegenerative disease subjects than in normal subjects. Therefore, detection of one or more of these markers in a person would provide useful information regarding the probability that the person may have a neurodegenerative disease.

The detection of a marker is then correlated with a probable diagnosis of neurodegenerative disease. In some embodiments, the detection of the mere presence or absence of a marker, without quantifying the amount thereof, is useful and can be correlated with a probable diagnosis of neurodegenerative disease. The measurement of markers may also involve quantifying the markers to correlate the detection of markers with a probable diagnosis of neurodegenerative disease. Thus, if the amount of the markers detected in a subject being tested is different compared to a control amount (i.e., higher or lower than the control, depending on the marker), then the subject being tested has a higher probability of having neurodegenerative disease.

The correlation may take into account the amount of the marker or markers in the sample compared to a control amount of the marker or markers (up or down regulation of the marker or markers) (e.g., in normal subjects or in subjects where neurodegenerative disease is undetectable). A control can be, e.g., the average or median amount of marker present in comparable samples of normal subjects in normal subjects or in subjects such as where neurodegenerative disease is undetectable. The control amount is measured under the same or substantially similar experimental conditions as in measuring the test amount. As a result, the control can be employed as a reference standard, where the normal phenotype is known, and each result can be compared to that standard, rather than re-running a control.

Accordingly, a marker profile may be obtained from a subject sample and compared to a reference marker profile obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of neurodegenerative disease status.

Any marker, individually, is useful in aiding in the determination of neurodegenerative disease status. First, the selected marker is detected in a subject sample using the methods described herein (e.g. mass spectrometry). Then, the result is compared with a control that distinguishes neurodegenerative disease status from non-neurodegenerative disease status. As is well understood in the art, the techniques can be adjusted to increase sensitivity or specificity of the diagnostic assay depending on the preference of the diagnostician.

While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, one method of the present invention provides for the measurement of more than one marker.

### Monitoring

Methods of qualifying neurodegenerative disease status in a subject are also useful in managing subject treatment based on neurodegenerative disease status. The invention provides for such methods where the markers (or specific combinations of markers) are measured before and again after subject management. In these cases, the methods are used to monitor the status of the neurodegenerative disease, e.g., response to neurodegenerative disease treatment, amelioration of the disease or progression of the disease.

For example, markers of the invention (e.g., GFAP, S100β, GLT-1/EAAT2) can be used to monitor a subject's response to certain treatments of human neurodegenerative disease. The level or function of a marker delineated herein may be measured before treatment, during treatment, or following the conclusion of a treatment regimen. Preferably, multiple measurements (e.g., 2, 3, 4, 5) are made at one or more of those times. Measurements are made, for example, using an immunoassay, microarray or quantitative real-time PCR to determine the expression profile of one or more markers (e.g., astrocytic proteins or polynucleotides). If desired, levels of an astrocytic marker are compared to levels of a non-astrocytic marker (e.g., EAAT1, OMP, MCT-4) to determine if the observed effect is astrocyte-specific. Such monitoring may be useful, for example, in assessing the efficacy of a particular drug in a patient. Therapeutics that increase the expression of an astrocytic polypeptide or polynucleotide are taken as particularly useful in the invention.

### Kits

In one aspect, the invention provides kits for monitoring and diagnosing neurodegenerative disease, wherein the kits can be used to detect the markers described herein. For example, the kits can be used to detect any one or more of the markers potentially differentially present in samples of neurodegenerative disease subjects vs. normal subjects (e.g., GFAP, S100β, GLT-1/EAAT2) or control proteins (e.g., EAAT1, OMP, MCT-4). If desired a kit of the invention include any one or more of the following: GFAP, S100β, MCT-4, EAAT2, EAAT1, OMP, glutamate receptors subtypes, and nerve growth factor receptors. The kits of the invention have many applications. For example, the kits can be used to differentiate if a subject has a neurodegenerative disease or has a negative diagnosis, thus aiding a neurodegenerative disease diagnosis. In another embodiment, the invention provides kits for aiding the diagnosis of neurodegenerative disease or the diagnosis of a specific type of neurodegenerative disease such as, for example, amyotrophic lateral sclerosis, muscular sclerosis, epilepsy, stroke, Huntington's disease, glioma, noise-induced hearing loss, neuropathic pain, obsessive-compulsive disorder, and mood disorders, such as depression. The kits can also be used to identify compounds that modulate expression of one or more of the herein-described markers in vitro or in vivo animal models for neurodegenerative disease.

The kits of the invention may include instructions for the assay, reagents, testing equipment (test tubes, reaction vessels, needles, syringes, etc.), standards for calibrating the assay, and/or equipment provided or used to conduct the assay. Reagents may include acids, bases, oxidizing agents, marker species. The instructions provided in a kit according to the invention may be directed to suitable operational parameters in the form of a label or a separate insert.

The kits may also include an adsorbent, wherein the adsorbent retains one or more markers selected from one or more of the markers described herein, and written instructions for use of the kit for detection of neurodegenerative disease. Such a kit could, for example, comprise: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. Accordingly, the kit could comprise (a) a DNA probe that specifically binds to a marker; and (b) a detection reagent. Such a kit could further comprise an eluant (as an alternative or in combination with instructions) or instructions for making an eluant, wherein the combination of the adsorbent and the eluant allows detection of the markers using gas phase ion spectrometry.

Optionally, the kit may further comprise a standard or control information so that the test sample can be compared with the control information standard to determine if the test amount of a marker detected in a sample is a diagnostic amount consistent with a diagnosis of neurodegenerative disease.

### Screening Assays

Physicians and scientists are increasingly aware that a subject's individual genetic profile (i.e., the unique set of markers expressed by a particular subject) can influence how the subject responds to particular therapeutic regimens. Therapeutic agents that are safe and effective for the treatment of certain subjects may not be safe and/or effective in all subjects. Methods that determine whether an agent is effective for the treatment of a particular subject are useful in allowing for personalized therapies. The invention provides methods for determining the efficacy of a therapeutic agent in a particular subject. Such methods may be carried out using cells obtained in an olfactory biopsy. In one embodiment, the method involves administering to a subject identified as having or having a propensity to develop a neurodegenerative disease a therapeutic agent and assaying the expression of astrocytic polypeptides (e.g., GFAP, S100β, GLT-1/EAAT2) expressed in cells of the subject obtained during an olfactory epithelium biopsy. The level or function of such polypeptides is compared to the level or function of such polypeptides present in a reference or present in the subject prior to treatment. If desired, an alteration in the expression of an astrocytic polypeptide is compared to expression of a non-astrocytic protein (e.g., EAAT1, OMP, MCT-4). Preferably, methods of the invention are used to identify therapeutic or prophylactic agents that alter the level or function of a polypeptide (e.g., EAAT2) whose level or biological activity is altered in a subject identified as having a neurodegenerative disease. An agent that increases the level of a marker of the invention (e.g., EAAT2) in cells of a subject is identified as effective for the treatment of that subject.

In another embodiment, the method involves obtaining tissues or cells from a subject (e.g., during an olfactory epithelium biopsy) and culturing such cells in vitro. The cells are then treated with a candidate agent and the level or function of an astrocytic polypeptide in the cells is compared to untreated corresponding control samples to characterize the efficacy of the agent. In another embodiment, the invention provides in vitro methods for the high-throughput low-cost screening of candidate compounds to identify those that modulate the expression of a set of astrocytic polypeptides. Knowing the identity of sequences that are differentially regulated in the presence and absence of a therapeutic agent provides for the identification of agents useful for the prevention or treatment of a neurodegenerative disease, as well as for providing insights into mechanisms of drug action.

Any number of methods are available for carrying out screening assays to identify new candidate agents that promote the expression of an astrocytic polypeptide whose expression or function is altered in a neurodegenerative disease. In one example, agents are added at varying concentrations to the culture medium of cultured cells expressing one of the nucleic acid sequences of the invention. Gene expression is then measured, for example, by microarray analysis, Northern blot analysis (Ausubel et al., supra), reverse transcriptase PCR, or quantitative real-time PCR using any appropriate fragment prepared from the nucleic acid molecule as a hybridization probe. The level of gene expression in the presence of the candidate compound is compared to the level measured in a control culture medium lacking the candidate molecule. A compound that promotes an increase in the expression of an astrocytic polynucleotide, or a functional equivalent thereof, is considered useful in the invention; such a molecule may be used, for example, as a therapeutic to treat a neurodegenerative disease in a human patient.

In another example, the effect of candidate agents may be measured at the level of polypeptide production using the same general approach and standard immunological techniques, such as Western blotting or immunoprecipitation with an antibody specific for an astrocytic polypeptide. For example, immunoassays may be used to detect or monitor the expression of at least one of the polypeptides of the invention in an organism. Polyclonal or monoclonal antibodies, (produced as described above) that are capable of binding to such a polypeptide may be used in any standard immunoassay format (e.g., ELISA, Western blot, or radioimmuno assay) to measure the level of the astrocytic polypeptide. In some embodiments, an agent that promotes an increase in the expression or biological activity of the polypeptide is considered particularly useful. Again, such a molecule may be used, for example, as a therapeutic to delay, ameliorate, or treat a neurodegenerative disease, or the symptoms of a neurodegenerative disease, in a human patient.

In yet another example, candidate agents may be screened for those that specifically bind to an astrocytic polypeptide. The efficacy of such a candidate agent is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention. In another embodiment, a candidate agent is tested for its ability to enhance the biological activity of a polypeptide described herein.

In another example, a nucleic acid described herein (e.g., a astrocytic polynucleotide) is expressed as a transcriptional or translational fusion with a detectable reporter, and expressed in an isolated cell (e.g., mammalian) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters (e.g., increases) the expression of the detectable reporter is a compound that is useful for the treatment of a neurodegenerative disease (e.g., amyotrophic lateral sclerosis, muscular sclerosis, epilepsy, stroke, Huntington's disease, and mood disorders, such as depression). In preferred embodiments, the candidate agent increases the expression of a reporter gene fused to an astrocytic polynucleotide.

In one particular example, a candidate agent that binds to an astrocytic polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the astrocytic polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Agents isolated by this method (or any other appropriate method) may, if desired, be further purified (e.g., by high performance liquid chromatography). In addition, these candidate agents may be tested for their ability to increase the activity of a astrocytic polypeptide. Agents isolated by this approach may also be used, for example, as therapeutics to treat a neurodegenerative disease in a human patient. Agents that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential agonists and antagonists include organic molecules, peptides, peptide mimetics, polypeptides, nucleic acids, and antibodies that bind to a nucleic acid sequence or polypeptide of the invention. For those nucleic acid sequences or polypeptides whose expression is decreased in a patient having a neurodegenerative disease, agonists would be particularly useful in the methods of the invention. For those nucleic acid molecules or polypeptides whose expression is increased in a patient having a neurodegenerative disease, antagonists would be particularly useful in the methods of the invention.

Each of the DNA sequences listed herein may also be used in the discovery and development of a therapeutic agent for the treatment of neurodegenerative diseases. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds that alter the expression or activity of an astrocytic polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to alter the activity or expression of an astrocytic polypeptide, further fractionation of the positive lead extract is necessary to isolate chemical constituents responsible for the observed effect. Thus, the goal of the extraction, fractionation, and purification process is the careful characterization and identification of a chemical entity within the crude extract that increases the activity of an astrocytic polypeptide. Methods of fractionation and purification of such heterogenous extracts are known in the art. If desired, compounds shown to be useful as therapeutics for the treatment of a human neurodegenerative disease are chemically modified according to methods known in the art.

### Pharmaceutical Compositions

Methods of the invention were used to identify ceftriaxone and thiamphenicol as agents that increase glutamate transporter EAAT2/GLT-1 function and gene expression. These agents also increased EAAT2/GLT-1 gene expression in nasal olfactory epithelium. Interestingly, ceftriaxone and thiamphenicol activated the transporter on a promoter level and increased transporter function in vitro and in vivo. Upregulation of glutamate transporter gene expression has been shown to protect against glutamate-mediated neuronal cell death, as seen in a large number of neurodegenerative diseases, including ALS, Huntington's disease, ischemia, and depression.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Alterations in the levels of specific polypeptides and/or polynucleotides has been associated with a variety of diseases. These disease-specific markers or biomarkers provide an opportunity to relate long term clinical outcome measures to immediate validated biochemical changes caused by drug therapy. For example, identification of a candidate compound that induces normalization of a biomarker associated with disease is likely to show efficacy in other measures of drug activity. This provides an efficient means of evaluating efficacy, relative to conventional means of measuring efficacy that rely assessing survival. Disease-specific markers are also useful for monitoring disease progression and assessing drug efficacy in clinical trials. Olfactory epithelial tissue expresses certain CNS markers (e.g., glutamate receptors subtypes, GFAP, nerve growth factor receptor, as well as neurotransmitters, such as GABA and glutamate) that are typically expressed only in neurons or glial cells. As reported in more detail below, EEAT2 is an astrocytic glutamate transporter that has been identified in olfactory epithelia. Measuring changes in gene expression levels provided an opportunity to monitor drug efficacy of EAAT2 upregulating compounds in rodents and humans.

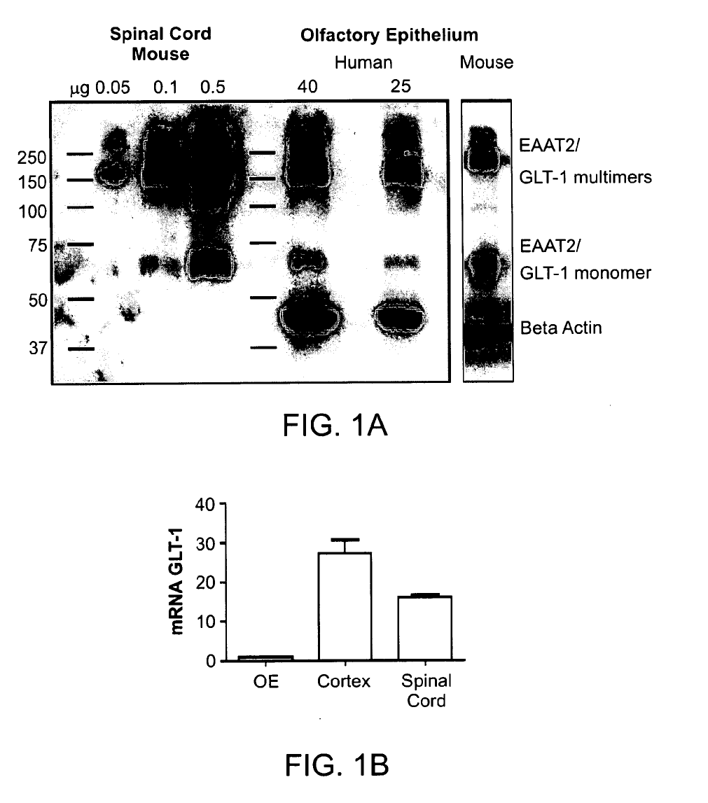

To study the expression of EAAT2 (in rodents GLT-1) in olfactory epithelium, mouse and human nasal tissue was analyzed for EAAT2/GLT-1 protein levels using standard immunoblotting techniques. Human olfactory mucosa was obtained via outpatient nasal biopsy, a low-risk procedure that is well established and that has no discernible adverse effects on any parameter, including olfactory function. Lysates of tissue samples were separated on SDS gels and compared with mouse spinal cord tissue lysates for their glutamate transporter expression levels (FIG. 1A). While expression levels in nasal tissue were much lower compared to levels in spinal cord tissue, both mouse and human olfactory epithelium showed similar immunoblot bands that stained positive for monomeric, as well as multimeric transporter complexes (FIG. 1A). Expression was also evaluated at the mRNA level using quantitative real time reverse transcriptase PCR (qRT-PCR) (FIG. 1B). These quantitative mRNA measurements confirmed the expression of EAAT2/GLT-1 in the nasal tissue.

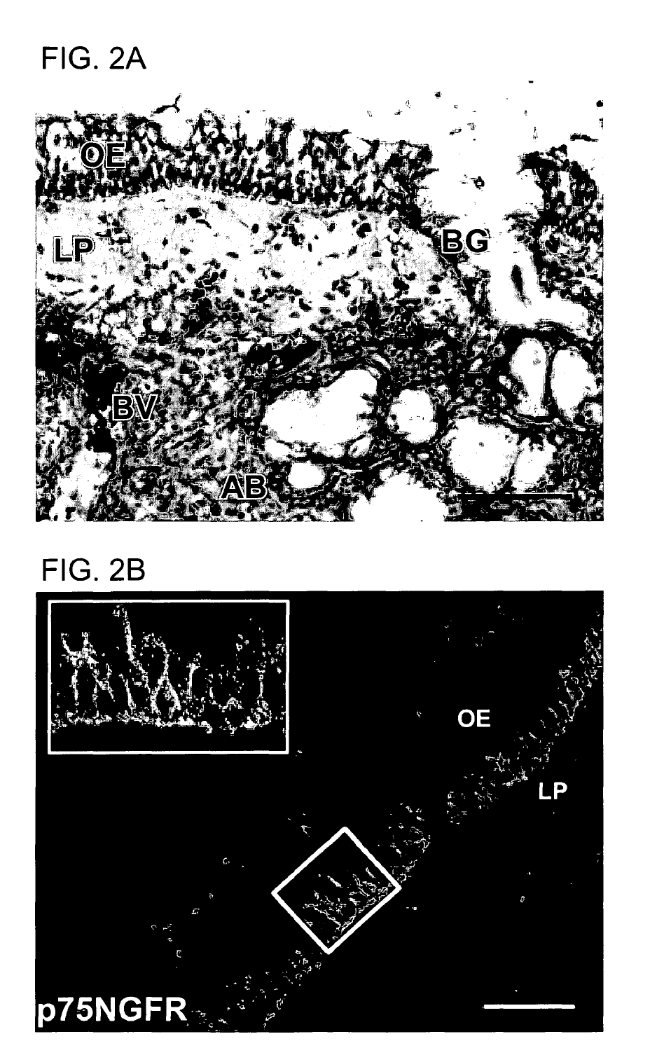

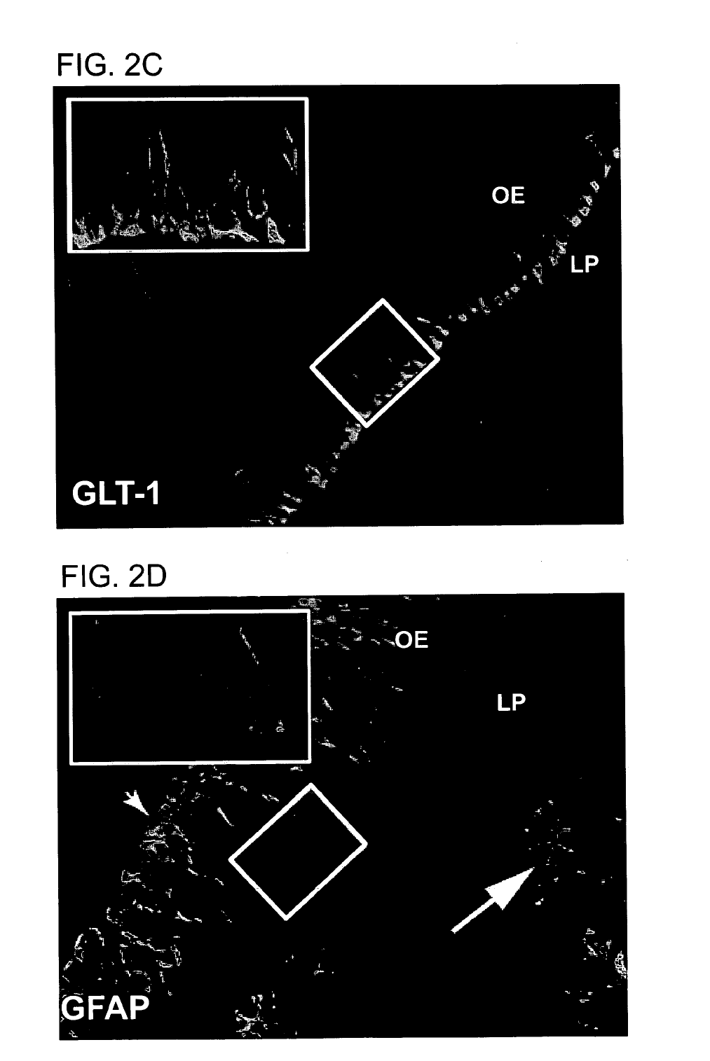

To determine which cells express glutamate transporters, human nasal olfactory epithelium tissue samples were fixed and immunostained. The olfactory mucosa is composed of the olfactory epithelium and the lamina propria (LP) (see FIG. 2A). The olfactory epithelium contains three basic cell types: olfactory receptor neurons (ORN), supporting cells (S) as well as basal cells (B). The supporting cells, also known as sustentacular cells, are believed to share similar functions with central nervous system glial cells. Sustentacular cell functions include insulating the olfactory receptor cells and providing guidance for the receptor cell axons. The basal cells consist of two subtypes, horizontal and global basal cells, which were identified as the neuroblast stem cells of the olfactory epithelium. The lamina propria contains the axons of the olfactory receptor neurons that are reaching towards the olfactory bulb. These axons are found in axon bundles surrounded by olfactory ensheathing cells and supported by connective tissue composed of fibroblasts, Bowman glands and blood vessels (see FIG. 2A).

Double labeling with antibodies to EAAT2 and the basal cell marker p75NGFR indicated co-localization of these two proteins in the basal cell layer of the olfactory epithelium (FIGS. 2B and 2C). EAAT2 was not detected in the supporting cells surrounding the olfactory receptor neurons or in the ensheathing cells of the lamina propria. The astrocytic marker GFAP (glial fibrillary acidic protein) was found in basal cells and olfactory ensheathing cells of the lamina propria. Similar results were reported by Au and Roskams (2003) in mouse olfactory epithelium (FIG. 2D).

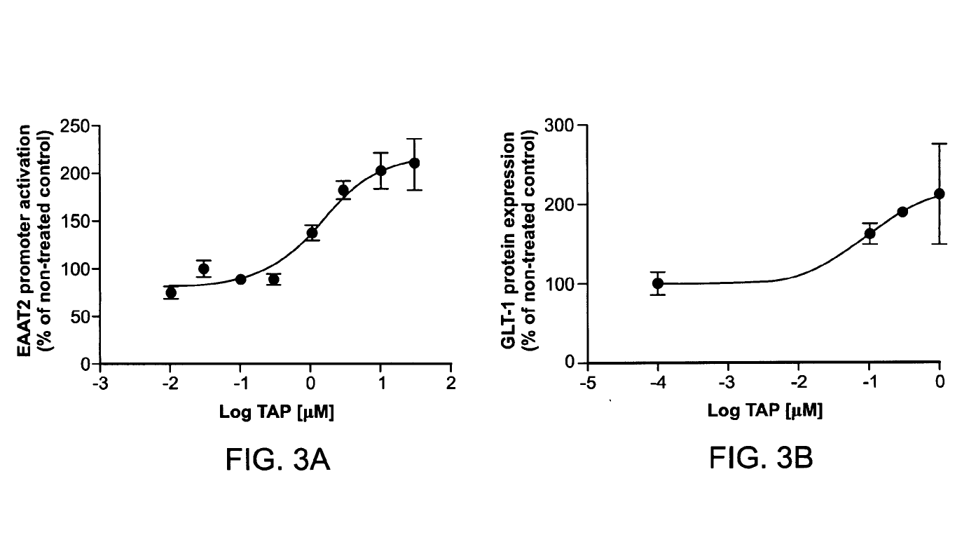

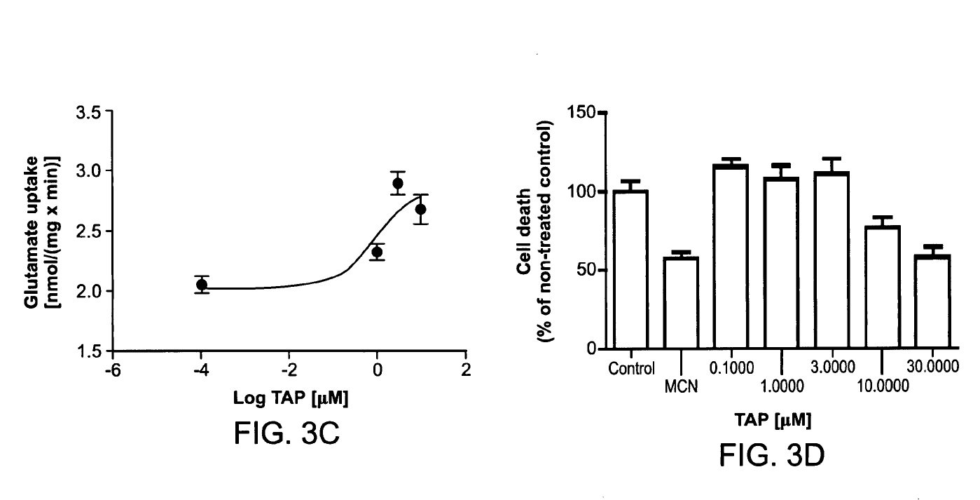

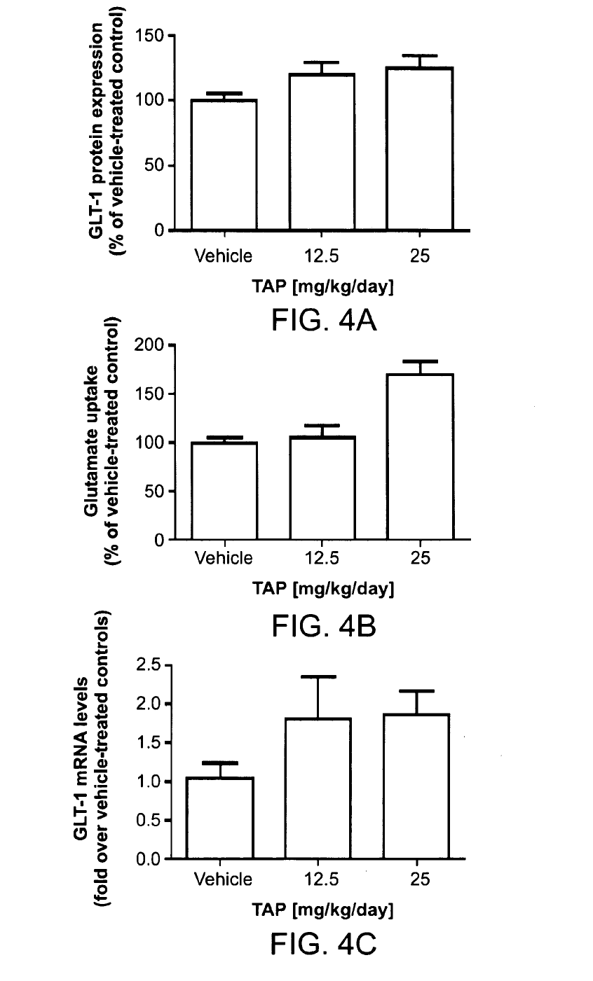

To determine whether the efficacy of glutamate transporter upregulating compounds could be detected in olfactory epithelium, changes in EAAT2 gene expression levels were monitored following treatment with compounds known to upregulate glutamate transporter gene expression. Ceftriaxone and Thiamphenicol are transporter upregulating compounds identified in a recent screen of FDA approved drugs for agents that increase glutamate transporter GLT-1/EAAT2 gene expression (Rothstein et al., Nature. 2005 Jan. 6; 433(7021):73-7). Follow up studies showed that ceftriaxone and thiamphenicol increase transcriptional activation as well as transporter function in vitro and in vivo ((Rothstein et al., Nature. 2005 Jan. 6; 433(7021):73-7) 1, FIGS. 3A-3D, 4A-4C). Upregulation of glutamate transporter gene expression has been suggested to protect against glutamate-mediated neuronal cell death. The neuroprotective properties of glutamate transporter upregulating compounds has since then been shown in a number of animal models of neurodegenerative diseases including ALS, HD, ischemia, and depression.

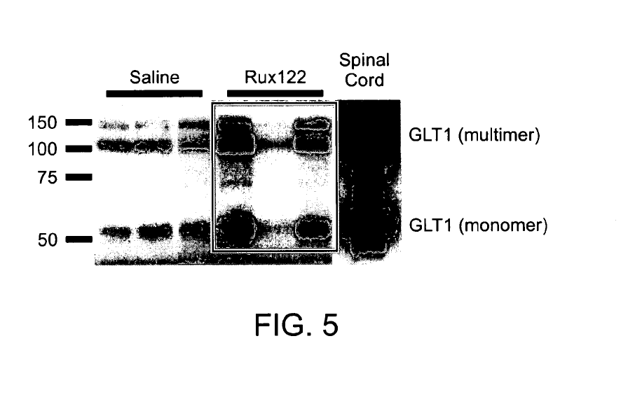

To test the use of nasal OE tissue as a measure for glutamate transporter upregulation, wild-type mice were treated with thiamphenicol for 14 days. Lysates of cortex, spinal cord and olfactory epithelium were analyzed for GLT-1 protein. Western blot analysis confirmed an upregulation of glutamate transporter protein GLT-1 in CNS tissues and nasal olfactory epithelium samples (FIG. 5). These results indicate that changes in olfactory epithelial tissue gene expression mirror changes occurring in the CNS.

To confirm that a similar upregulation can be followed in nasal olfactory epithelium of humans, nasal biopsy tissue samples from healthy volunteers taking thiamphenicol (TAP) for 14 days were analyzed in a double blinded placebo controlled inpatient trial. The study was designed to obtain pharmacokinetic and safety data on TAP and to collect drug-induced pharmacodynamic data in the form of EAAT2 mRNA levels from nasal olfactory epithelium. Thirty-four normal volunteers were divided into three groups: placebo (N=6), TAP 750 mg administered orally once a day (N=12) and TAP 1500 mg administered orally once a day (N=12). Nasal biopsies were taken on day −1 (before drug treatment) and day 14. Plasma pharmacokinetics were done on day 1 and 13 and cerebrospinal fluid samples were taken on day 13 to study the levels of TAP in the brain. In addition to EAAT2, the mRNA levels of the following genes were measured: GFAP (astrocytic expression in CNS), EAAT1 (astrocytic expression in CNS, expressed in precursor neuronal cells in olfactory epithelium), OMP (neuronal expression in olfactory epithelium and CNS), and MCT-4. Validation of the measurements of mRNA levels in human olfactory epithelium samples obtained from normal volunteers revealed a low inter- and intra-patient variability. The intra-patient variability was obtained by comparing mRNA levels obtained from the left and right side of the nose of each volunteer. This was important since the drug study was designed to obtain a biopsy from one side of the nose before drug treatment and from the opposite side of the nose after drug treatment. This allowed each person to serve as their own control and provided an opportunity to follow changes in mRNA levels within individuals.

TAP was well tolerated at every dose and no serious adverse side-effects were reported. The pharmacokinetic profile revealed dose-related plasma concentrations, no drug accumulation, and an average half life of 4-6 hours (see Table 1)

TABLE 1

| | TAP | TAP | TAP | TAP |
|---|---|---|---|---|
| | Day 1 | Day 13 | Day 1 | Day 13 |
| Dose | 750 mg/day | 750 mg/day | 1500 mg/day | 1500 mg/day |
| N | 12 | 12 | 12 | 12 |
| Cmax | 6306 ± | 6553 ± | 10713 ± | 11045 ± |
| (ng/ml) | 1508 | 1249 | 2380 | 2051 |
| Tmax | 2.0 (1-4) | 2.0 (1-6) | 2.5 (2-6) | 2.0 (1-6) |
| median(range) | | | | |
| AUC | 42566 ± | 51108 ± | 88047 ± | 96740 ± |
| | 6700 | 6427 | 17080 | 27877 |
| T ½ | 4.1 ± 0.8 | 4.8 ± 0.6 | 4.2 ± 0.5 | 5.7 ± 1.0 |

Table 1 provides a summary of human pharmacokinetics of Thiamphenicol. Healthy volunteers were dosed with either 750 mg/kg or 1500 mg/kg/day. Basic pharmacokinetic analyses (Cmax, Tmax, area under the curve (AUC) and half life (T1/2) were done on day 1 and day 13 of drug treatment. The obtained half life was comparable with data found in the literature of previous clinical trials using Thiamphenicol.

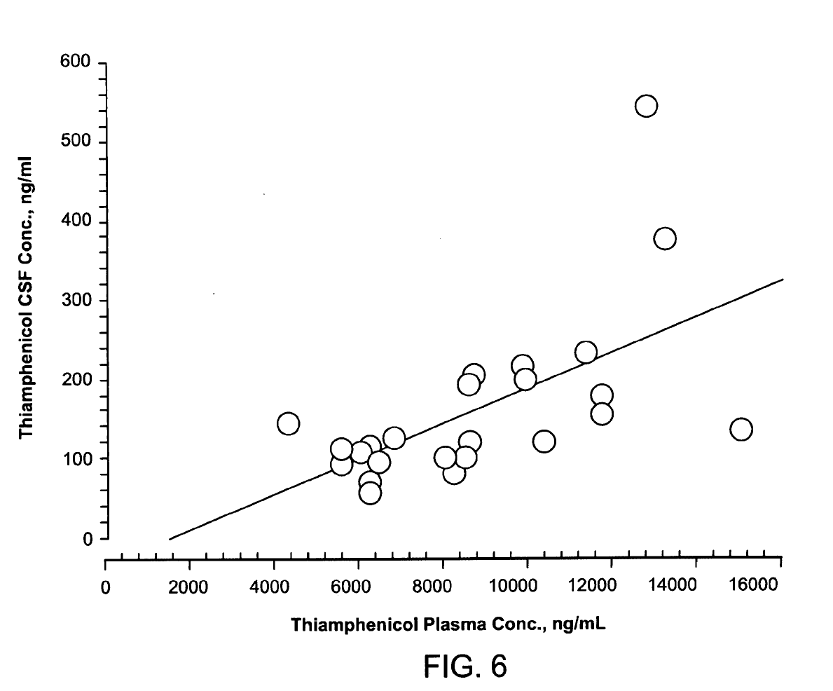

Furthermore, TAP dose-dependently penetrated into the brain with cerebrospinal fluid levels in ng/ml at 101.8+/−23.1 at 750 mg/day and 220.2+/−123.5 at 1500 mg/day (FIG. 6). These levels correlated well with in vitro levels used to increase GLT-1 protein in dissociated mouse mixed cultures and showed a linear correlation when compared to plasma levels. Accordingly, the invention provides methods for providing TAP at 750 mg/day or 1500 mg/day.

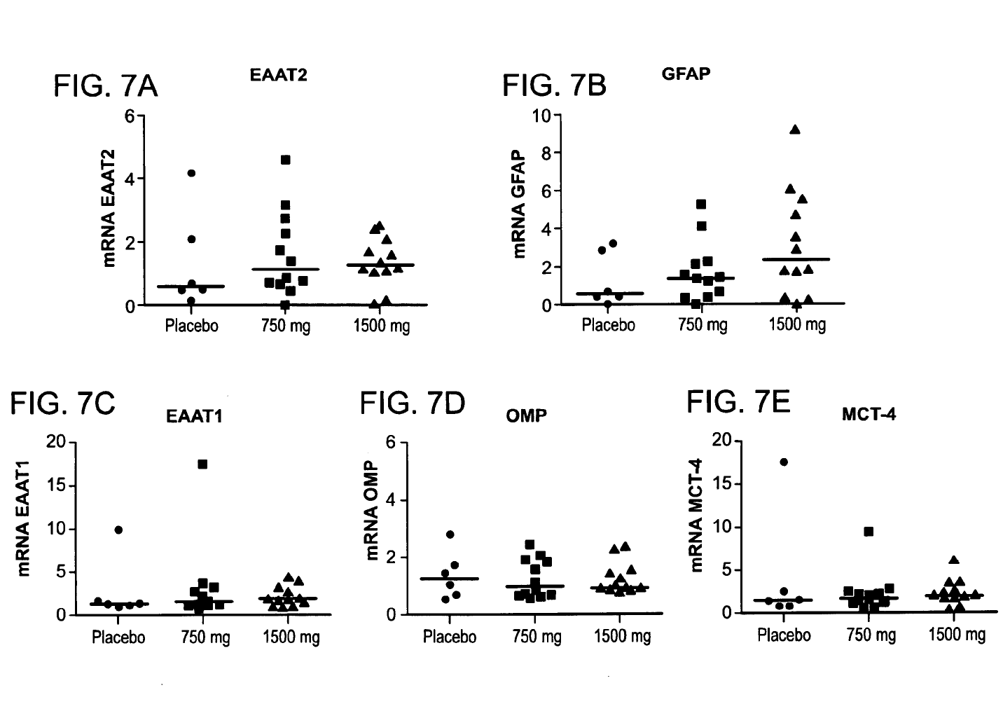

Analysis of the nasal biopsy samples before and after drug treatment revealed a dose-dependent positive trend towards an increase in mRNA levels of both astrocytic genes, EAAT2 and GFAP (FIGS. 7A-7E). Non-astrocytic genes OMP, EAAT1 and MCT-4 showed no difference in mRNA levels due to drug treatment (FIG. 7A-7E), confirming a specificity of drug-induced increases in astrocytic gene expression. The positive trend in mRNA levels indicates that TAP is likely to be useful for increasing the expression of astrocytic changes, and also indicates that nasal biopsy samples are useful for monitoring alterations in astrocytic biomarkers.

These studies are the first to indicate that the glutamate transporter protein EAAT2/GLT-1 is expressed in nasal epithelial mucosa. EAAT2/GLT-1 was detected in p75-positive basal cells located in the region near the lamina propria. Based on animal studies, basal cells are considered the neuroblast stem cells of the olfactory neuroepithelium. Surprisingly, EAAT2/GLT-1 was not detected in olfactory ensheathing cells located in the lamina propria of the olfactory mucosa. In contrast, the astrocytic protein GFAP was detected in ensheathing cells.

Mice and humans treated with glutamate transporter upregulating agents both indicated that alterations in astrocytic protein levels in nasal tissue mirror changes in gene expression occurring in the CNS. This finding indicates that biomarkers present in olfactory epithelia can be used to monitor drug-efficacy in the CNS. This is likely to be particularly useful in monitoring changes in glutamate transporter-targeted therapy and for monitoring disease progression by measuring glutamate transporter gene expression in ALS patients. Data obtained using an ALS animal model (SOD1 mutant mice) indicated that reductions in GLT-1 mRNA in the nasal mucosa resemble those observed in murine spinal cords.

The results reported herein were obtained using the following materials and methods.

### Isolation of Nasal Olfactory Epithelial Proteins in Mice

Mouse nasal turbinates were dissected on dry ice and stored at −80° C. For protein isolation, nasal tissue was homogenized in Tris buffer and solubilized in 0.1% SDS followed by sonication. Tissue lysate was separated on an SDS gel and immunoblotted for astrocytic proteins. For mRNA isolation nasal tissue was homogenized using a Polytron tissue grinder and RNA was isolated using standard methods and commercially available reagents, RNA Easy kit (Qiagen) according to the manufacturer's protocol. RNA was stored at −80° C. until RT-qPCR analysis.

### Nasal Biopsies in Human Volunteers

This study was approved by the Institutional Review Board of the Johns Hopkins University, the University of Maryland and the Review Board of the Belgium CRO overseeing the nasal biopsies in drug-treated healthy volunteers. Volunteers were selected randomly to participate in the study of the approved protocol after written informed consent was obtained. The technique for a safe biopsy in the living was developed in 1982 (Lanza, D. C. et al. The effect of human olfactory biopsy on olfaction: a preliminary report. Laryngoscope 104, 837-840 (1994), which is incorporated by reference in its entirety and since then has been used continuously. No negative effects on olfactory function have been reported (Jafek, B. W., Murrow, B., Michaels, R., Restrepo, D. & Linschoten, M. Biopsies of human olfactory epithelium. Chem. Senses 27, 623-628 (2002), Lovell, M. A., Jafek, B. W., Moran, D. T. & Rowley, J. C., III Biopsy of human olfactory mucosa. An instrument and a technique. Arch. Otolaryngol. 108, 247-249 (1982)). An increasing number of laboratories have performed this technique using local anesthesia to study olfactory physiology as well as dysfunction in both patients as well as an even larger number of healthy controls⁴⁻¹¹.

Human nasal biopsies were performed by otolaryngologists specializing in sinus surgery. The procedure took approximately 30 minutes to perform and subjects were observed in the clinic for 15 to 30 minutes after biopsy completion. Local anesthesia was used for the procedure. Local anesthesia was applied to the nasal cavity. Lidocaine liquid 4% (Xylocaine®) and oxymetazoline HCl 0.05% (Afrin®) was sprayed in the nose. Then, if needed, 1% lidocaine with 1/100,000 epinephrine was injected to provide both anesthesia and vasoconstriction.

The procedure was performed under endoscopic control and used either a small curette or biting forceps for tissue removal. To avoid trauma to the cribriform plate, the biopsies were usually taken from the upper nasal septum. Occasionally, a small piece of superior turbinate (which is usually lined with olfactory tissue) was removed from the lateral nasal wall. The tissue was removed from either the front or the back of the olfactory cleft, and sometimes both. Up to two blocks of tissue (approximately 2 mm each) were collected from each side of the nose. Persons who have had the biopsy generally have no post-biopsy problems except for mild nasal- or head-ache for a few hours, and possibly a small amount of spotting of blood from the nose. Subjects were instructed not to blow their nose and not to perform any strenuous physical activity for 24 hours after the procedure.

### Study Design for Drug Treatment of Healthy Volunteers (Inpatient)

Healthy volunteers underwent a nasal biopsy on day −1 on one side of the nose only. R UX122 was administered in 3 groups (placebo, 750 mg/day, 1500 mg/day) starting day 1 to day 14. Blood samples were taken throughout the trial period to obtain basic pharmacokinetic analyses as well as to monitor for safety and tolerability. Every patient underwent a spinal tap to collect CSF on day 13. On day 14, a nasal biopsy was done on the other side of the nose. Patients were observed for another 2-3 hrs before being released.

### Quantitative RT-PCR

Total RNA was reversed transcribed using Applied Biosystems' high capacity cDNA archive kit. The quantitative real time RT-PCR assay was performed using the iCycler iQ Real-Time Detection System from BioRad. TaqMan Probes, which allow for specific mRNA amplification, were purchased from Applied Biosystems.

### Immunoblotting

EAAT2/GLT-1 protein was detected by standard immunoblotting techniques with anti-GLT-1 antibody. Human EAAT2 was recognized by anti-GLT-1 antibody (ref????). Briefly, tissue was lysed in SDS-containing Tris buffer and semi-quantitatively analyzed by SDS-PAGE Western Blot analysis.

### Immunohistochemistry

Fresh nasal tissue was fixed with 4% paraformaldehyde in PBS for 1 hour at room temperature. After rinsing with PBS, tissue was cryoprotected in 30% sucrose in PBS until the tissue sank. The tissue was subsequently embedded in tissue freezing medium (Triangle Biomedical Sciences, INC.) for cryostat sectioning. 12 μm sections of the nasal tissue were prepared for immunofluorescence or DAB staining. Sections were post fixed with 4% paraformaldehyde in phosphate buffered saline (PBS) and incubated in blocking buffer (10% normal goat serum, 0.2% Triton X-100 in PBS) for 60 minutes at room temperature, and then incubated with primary antibodies against GLT1 (rabbit, 1:200), p75 NGFR (mouse, 1:500, Sigma) or GFAP (rabbit, 1:100, DAKO) diluted in 3% normal goat serum, 0.2% Triton X-100 in PBS overnight at 4 C. For immunofluorescence, Alexa 594/647 goat anti-mouse or anti-rabbit IgG (1:1000, Invitrogen) was applied as a secondary antibody for 2 hours at room temperature, followed by nuclear counter staining with Hoechst 33342 (2 μg/ml, Invitrogen). For 3,3′-diaminobenzidine (DAB) staining, biotinylated goat anti-mouse or anti-rabbit IgG (1:200, Vector Laboratories) was applied to the tissue sections for 60 minutes at room temperature, followed by avidin-biotin-peroxidase complex (Vectastain ABC kit, Vector Laboratories), according to the manufacturer's protocol. Immunoreactivity was visualized using DAB (Vector Laboratories) as a chromogen. Sections were then counterstained with Gill's hematoxylin. All double staining was performed sequentially. Confocal images of stained tissues were taken using a Zeiss LSM510 or 710 Meta.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for identifying a subject as having or having a propensity to develop a disease affecting the central nervous system, the method comprising
(a) obtaining a nasal tissue sample from the subject; and
(b) detecting an alteration in the level or function of an astrocytic polypeptide or polynucleotide in the tissue sample relative to a reference, thereby identifying the subject as having or having a propensity to develop a disease affecting the central nervous system.

2. A method for identifying a subject as having or having a propensity to develop a neurodegenerative disease associated with astrocyte dysfunction, the method comprising
(a) obtaining a nasal tissue sample from the subject; and
(b) detecting an alteration in the level or function of an EAAT2 or GFAP polypeptide or polynucleotide in the tissue sample relative to a reference, thereby identifying the subject as having or having a propensity to develop a neurodegenerative disease associated with astrocyte dysfunction.

3. A method for diagnosing a subject as having or having a propensity to develop amyotrophic lateral sclerosis, the method comprising
(a) obtaining a nasal tissue sample from the subject; and
(b) detecting a reduction in the level or function of an EA/T2 polypeptide or polynucleotide in the tissue sample relative to a reference, thereby identifying the subject as having or having a propensity to develop amyotrophic lateral sclerosis.

4. The method of claim 3, further comprising identifying an increase in the level of GFAP polypeptide or polynucleotide in the tissue sample relative to a reference.

5. A method for monitoring a disease affecting the central nervous system in a subject, the method comprising
(a) obtaining a nasal tissue sample from the subject; and
(b) detecting an alteration in the level or function of an astrocytic polypeptide or polynucleotide in the tissue sample relative to a reference.

6. The method of claim 5, wherein the reference is a subject sample obtained prior to treatment or at an earlier time point.

7. The method of claim 5, wherein the astrocytic polypeptide or polynucleotide is an EAAT2 polypeptide or polynucleotide.

8. The method of claim 5, wherein a reduction in EAAT2 expression relative to a reference indicates progression of the disease, and an increase in EAAT2 level indicates amelioration of the disease.

9. A method for detecting an agent's therapeutic efficacy in a subject having a disease affecting the central nervous system, the method comprising
(a) obtaining a nasal tissue sample from the subject; and
(b) detecting an alteration in the level or function of an astrocytic polypeptide or polynucleotide in the tissue sample following treatment relative to a reference.

10. The method of claim 5, wherein the reference was obtained from the subject prior to treatment or at an earlier time point during treatment.

11. The method of claim 9, wherein the astrocytic polypeptide or polynucleotide is an EAAT2 or GFAP polypeptide or polynucleotide.

12. The method of claim 9, wherein a reduction in EAAT2 expression or function relative to a reference indicates said agent lack's efficacy, and an increase in said level or function indicates that said agent has therapeutic efficacy in said subject

13. The method of claim 1 any one of claims 1-12, wherein the tissue sample is obtained during an olfactory epithelial biopsy.

14-18. (canceled)

19. The method of claim 1, wherein the polypeptide or polynucleotide is EAAT2.

20. The method of claim 1, wherein the disease is selected from the group consisting of amyotrophic lateral sclerosis, multiple sclerosis, epilepsy, stroke, Huntington's disease, Noise-induced hearing loss, glioma, Parkinson's disease, obsessive-compulsive disorder, neuropathic pain, depression, and a mood disorder.

21-23. (canceled)

24. The method of claim 1, wherein the subject is a human.

25. A method of identifying a candidate compound that ameliorates a neurodegenerative disorder, said method comprising contacting an olfactory tissue cell that expresses an astrocytic polypeptide or polynucleotide with a candidate compound, and comparing the level of expression in said cell relative to the level present in a reference, wherein a compound that alters the level of said astrocytic polypeptide or polynucleotide is identified as ameliorating a neurodegenerative disorder, or
A method of assessing the efficacy of a treatment for a neurodegenerative disease in a subject, the method comprising
(a) measuring an astrocytic marker in an olfactory tissue cell obtained from the subject;
(b) contacting the olfactory tissue cell with a candidate agent; and
(c) detecting an alteration in the marker level, wherein an alteration in the level of the marker is an indication that the treatment is efficacious for treating a neurodegenerative disease in the subject, or
A method of assessing the efficacy of a treatment for a neurodegenerative disease in a subject, the method comprising comparing: (i) the level of an astrocytic marker measured in a first olfactory tissue cell obtained from the subject before the treatment has been administered to the subject; and (ii) the level of the marker in a second olfactory epithelium biopsy sample obtained from the subject after the treatment has been administered to the subject, wherein an alteration in the level of the marker in the second sample relative to the first sample is an indication that the treatment is efficacious for treating a neurodegenerative disease in the subject, or
A method of identifying a candidate compound that ameliorates a central nervous system disease or disorder, said method comprising contacting olfactory tissue cell that expresses an astrocytic polypeptide or polynucleotide with a candidate compound, and comparing the level of expression of said polypeptide or polynucleotide in said cell contacted by said candidate compound with the level of expression in a corresponding control cell not contacted by said candidate compound, wherein an alteration in expression of said polypeptide or polynucleotide identifies said candidate compound as a candidate compound that ameliorates a central nervous system disease or disorder.

26-42. (canceled)
